(19) United States
(12) Patent Application Publication
(10) Pub. No.: US 2018/0155391 A1
(43) Pub. Date: Jun. 7, 2018

(54) Method for extracting proteins and functional lipid from soybean liquid by directional continuous catalysis with nano magnetic immobilized enzymes

(71) Applicant: Northeast Agricultural University, Harbin (CN)

(72) Inventors: Lianzhou JIANG, Harbin (CN); Dianyu YU, Harbin (CN); Lili ZHANG, Harbin (CN); Jianjun CHENG, Harbin (CN); Liqi WANG, Harbin (CN); Guidong XIE, Harbin (CN); Xu ZHANG, Harbin (CN); Xin LIU, Harbin (CN); Qing ZHANG, Harbin (CN); Zhongbin LI, Harbin (CN)

(21) Appl. No.: 15/886,829

(22) Filed: Feb. 1, 2018

Related U.S. Application Data
(63) Continuation of application No. PCT/CN2015/000549, filed on Aug. 3, 2015.

(51) Int. Cl. C07K 4/10; C07K 14/415; A01H 5/08; C12N 9/24; C11B 1/04; B01J 8/42

(52) U.S. Cl. CPC C07K 4/10; C07K 14/415; B01J 8/42; C12N 9/24; C11B 1/04; A01H 5/0831

(57) ABSTRACT

The present invention relates to fields of continuous directional catalysis of soybean slurry to extract protein and functional oil with nano-magnetic immobilized enzymes. The present invention discloses soybean slurry as raw material and continuous mobile phase, and nano-magnetic immobilized enzymes as stationary phase applied in three-phase magnetic fluidized bed. The present invention provides a method of enzymatic disrupting cell walls by nano-magnetic immobilized enzyme cocktail including cellulose, pectinase and alkaline protease. The method can he used to break down lipoprotein, lipopolysaccharide complex and break lipoprotein film on the surface of lipoprotein to release the lipid. The present invention also provides a method to catalyze phospholipid using nano-magnetic immobilized phospholipase to change the polarity of phospholipids and control liquid emulsification phenomenon. The enzymes disclosed in the present invention do not interfere with each other. The preparation method can be used to catalyze soybean slurry continuously using the nano-magnetic immobilized enzymes to extract protein and functional lipid simultaneously.

### TECHNICAL FIELD

The present disclosure relates to fields of soybean protein and lipid extraction, and more specifically to a method for nano-magnetic immobilized enzymes to continuously and directionally catalyze soybean slurry for the extraction of protein and functional lipid.

### BACKGROUND

Soybean is a rich protein leguminous plant with up to 40% protein content. The amino acid composition is similar to the amino acid composition of dairy products and contains all of the essential amino acids required for human being. Soybean contains up to 20% lipid and 3% phospholipids. The traditional extraction method of soybean lipid is solvent extraction method. The lipid yield can reach more than 99.5%. However, the protein is severely denaturized, and the toxic and harmful solvent used in the method causes several problems. Aqueous enzymatic extraction of lipid method is a new lipid extraction method, which in agreement with the current development concept of green production. The enzyme is applied to hydrolyze the protein within the soybean cells, in order to release the lipid with an lipid yield near 60%. The use of alkaline protease and neutral protease in the method can reach an lipid extraction rate of up to 66%. However, the enzymes in the current aqueous enzymatic extraction method inevitably interact with each other causing severe problems. The enzymatic hydrolysis efficiency is therefore lowered and the substrate is highly emulsified.

### SUMMARY

In order to solve a problem of nano-magnetic immobilized enzyme to continuously and directionally catalyze soybean slurry extraction protein and functional lipid. The description is as follows: The soybean slurry prepared by using crushed soybeans is used as a raw material; the nano-magnetic immobilized cellulose, the nano magnetic immobilized pectinase, the nano magnetic immobilized alkaline protease, the nano magnetic immobilized phospholipase C, the nano magnetic immobilization Phospholipase A1 is used in the respective three-phase magnetic fluidized bed, as a stationary phase, so that it was magnetically stable fluidized state distribution to soybean slurry as a continuous mobile phase, soybean slurry through different types of magnetic immobilized enzyme where the magnetic fluidized bed reactor, full access to the catalytic soybean feed substrate, nano-magnetic immobilized cellulase, nano-magnetic immobilized pectinase, nano-magnetic immobilized alkaline protease to break cell wall, decompose lipoprotein, lipopolysaccharide complex and destroy the surface of lipoprotein lipoprotein membrane, so that lipid out, use nano-magnetic immobilized phospholipase treatment of phospholipids to phospholipid polarity weakened by soybean phospholipase C enzymatic hydrolysis of lecithin (PC) and cephalin (PE), can produce functional characteristics of diglycerides (DAG) to Phospholipase C enzymatic hydrolysis of soybean PC solution, the enzymatic hydrolysis process shown in FIG. 1, through a small amount of phospholipase A1 enzymatic hydrolysis of soybean phospholipids to phospholipase A1 digest soybean PA (phosphatidic acid), the enzymatic hydrolysis process shown in FIG. 2, making the phospholipid polarity increased, the formation of the system easy Promote the reaction of phospholipase C, no matter which kind of enzyme mainly makes the phospholipid lose the emulsifying property, and uses the double set of enzymolysis process under the condition that no mechanical stirring is needed, thus realizing the nano-magnetic immobilized enzyme to continuously and continuously catalyze the soybean material extraction protein and the function Continuous production of lipid.

To be specifically, the description relates to a method for nano-magnetic immobilized enzyme to continuously and directionally catalyze soybean slurry extraction protein and functional lipid. To be more specifically, the description relates to the immobilized enzymes with magnetic nanoparticle (nano-magnetic immobilized cellulase, nano-magnetic immobilized pectinase, nano-magnetic immobilized alkaline protease, nano-magnetic immobilized phospholipase C, nano-magnetic immobilized phospholipase A1) were applied in gas-liquid-solid three-phase magnetic fluidized bed reactor. Five-order-three-phase fluidized bed reaction system is composed of three-phase magnetic fluidized bed, soybean material tank, pH regulating tank, power plant. The nano-magnetic immobilized enzyme in a three-phase magnetic fluidized bed continuous catalytic soybean slurry extraction protein and functional lipid process shown in FIG. 3, FIG. 4, firstly, the nano-magnetic immobilized enzymes were added in fluidized bed reactor as stationary phase and soybean was used as mobile phase. The nano-magnetic immobilized enzymes were to assume the magnetic steady and flow distribution by adjusting the reaction operation parameters and then reacted with the corresponding directional. According to the properties of enzymolysis of henano-magnetic immobilized enzymes, the effect of enzymolysis was increased by adjusting the order and reaction conditions to obtain soybean protein and the functional lipids which rich in DAG.

The invention has the advantages of using the nano-magnetic immobilized enzymes with the advantages of high thermal stability and tolerable pH range in a three-phase magnetic fluidized bed, and the enzymatic hydrolysis efficiency is improved, and especially each three-phase magnetic flow the best reaction conditions can be kept in the reactor, so that each kind of nano-magnetic immobilized enzyme can catalyze the enzymatic hydrolysis in an independent environment so as to realize the nano-magnetic immobilized enzyme to continuously and directionally catalyze the extraction of the soybean slurry protein and functional lipid, while nano-magnetic immobilized enzyme regeneration, make it reuse, reduce production costs.

### BRIEF DESCRIPTION OF THE DRAWINGS

FIG. 1. Phospholipase C digests PC-generated DAG from soybean feed.

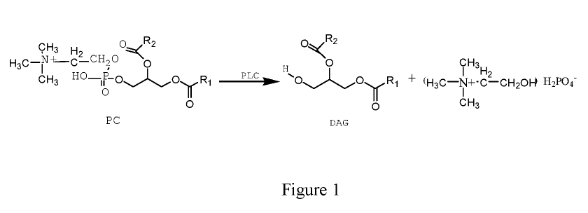

FIG. 2. Phospholipase A1 enzymatic hydrolysis of soy PA process.

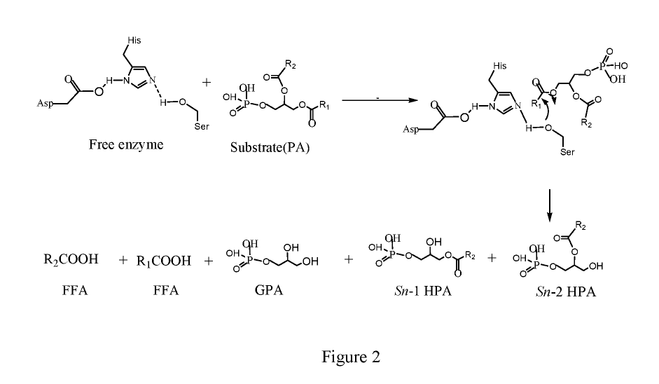

FIG. 3. Nano magnetic immobilized enzyme in a three-phase magnetic fluidized bed continuous catalytic soybean extract liquid protein and functional lipid process.

FIG. 4. Nano magnetic immobilized enzyme in a three-phase magnetic fluidized bed continuous catalytic soybean extract liquid protein and functional lipid process.

Nano-magnetic immobilized enzyme particles in the three-phase magnetic fluidized bed fluidization process:

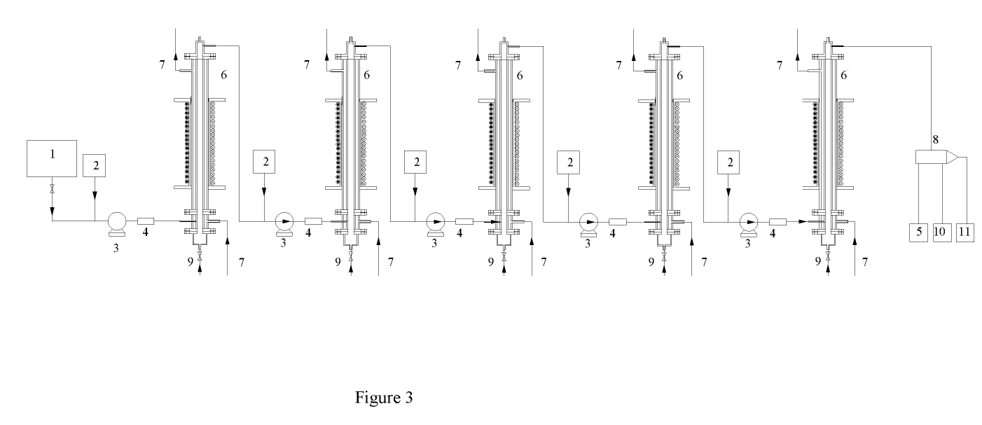

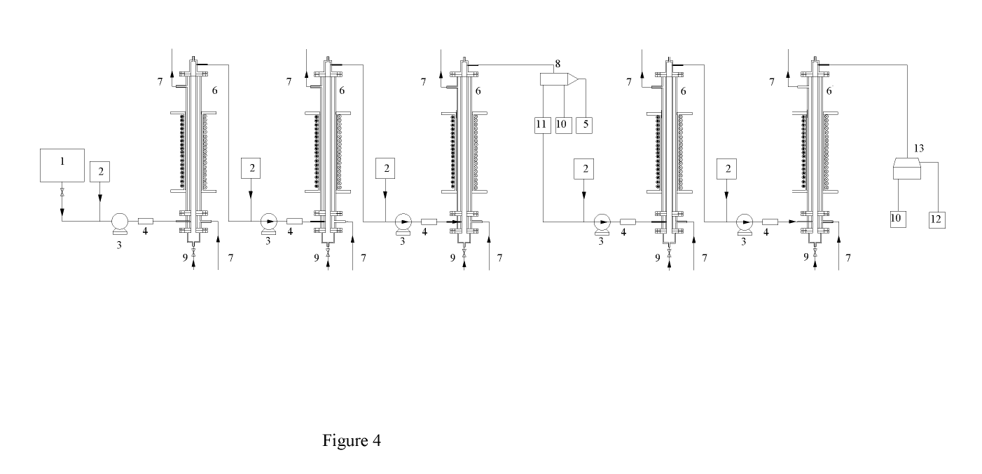

The soybean slurry is sent to a five-order-three-phase magnetic fluidized bed reaction system for self-circulation, and the DC power source is turned on, the nano-magnetic immobilized cellulase, the nano magnetic immobilized pectinase, the nano-magnetic immobilized alkaline protease, Nano-magnetic immobilized phospholipase C and nano-magnetic immobilized phospholipase A1 particles from the magnetic fluidized bed were added to the appropriate level of the port three-phase magnetic fluidized bed, open the air compressor outlet valve, open the hot water circulation system, to ensure that the reaction temperature of the system, by adjusting the operating parameters, so that nano-magnetite particles in the five three-phase magnetic fluidized bed reaction system to achieve fluidization. Before the enzyme solution flows into each bed of the fluidized bed, the optimal pH value of each magnetite reaction is adjusted. FIG. 4 is different from FIG. 3 in that the three-phase three-phase magnetic fluidized bed out of the enzyme solution obtained after centrifugation of the residue, enzyme solution and emulsion, the emulsion continued the follow-up operation, the other steps are the same.

### DETAILED DESCRIPTION OF THE INVENTION

### Embodiment 1

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 0.10 g/kg, and reaction duration of 4.0 h to hydrolysis pectin. Thirdly, the soybean slurry was transferred in the third order of three-phase magnetic fluidized bed reactor to continue the reaction. The mixture was adjusted pH to 9.5, temperature of 55° C. The reaction of the third order is: nano-immobilized magnetic alkaline protease dosage of 0.10 g/kg, the reaction duration of 4.0 h. Fourthly, the above soybean slurry was heated to 60° C. followed by adjusting pH to 7.0 using a buffer, and then the mixture was transferred in the fourth order of three-phase fluidized bed reactor to generate DAG from PC and PE. The reaction of the fourth order is: nano-immobilized magnetic phospholipase C dosage of 0.80 g/kg, the reaction duration of 4.5 h. Fifthly, the soybean slurry was transferred in the fifth order of three-phase magnetic fluidized bed reactor to continue the reaction. The mixture was adjusted pH to 6.5, temperature of 60° C., immobilized phospholipase A1 dosage of 0.40 g/kg, and reaction duration of 3.4 h. After completion of all other reaction steps, the mixture from the fifth order of three-phase fluidized bed reactor was centrifugated to obtain lipid, skim and residues. In the total skim, the protein yield was 94%, the lipid yield was 96% and the content of DAG in lipid was 1.5%. Other factors are optimized using the central combination design experimental methods, and the design and results are shown in Table 1. As seen from Table 1, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

### Embodiment 2

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 0.10 g/kg, and reaction duration of 4.0 h to hydrolysis pectin. Thirdly, the above soybean slurry was heated to 60° C. followed by adjusting pH to 7.0 using a buffer, and then the mixture was transferred in the third order of three-phase fluidized bed reactor to generate DAG from PC and PE. The reaction of the third order is: nano-immobilized magnetic phospholipase C dosage of 0.90 g/kg, the reaction duration of 4.5 h. Fourthly, the soybean slurry was transferred in the fourth order of three-phase magnetic fluidized bed reactor to continue the reaction. The mixture was adjusted pH to 6.5, temperature of 60° C., immobilized phospholipase A1 dosage of 0.50 g/kg, and reaction duration of 3.4 h. Fifthly, the soybean slurry was transferred in the fifth order of three-phase magnetic fluidized bed reactor to continue the reaction. The mixture was adjusted pH to 9.5, temperature of 55° C. The reaction of the fifth order is: nano-immobilized magnetic alkaline protease dosage of 0.12 g/kg, the reaction duration of 4.0 h. After completion of all other reaction steps, the mixture from the fifth order of three-phase fluidized bed reactor was centrifugated to obtain lipid, skim and residues. In the total skim, the protein yield was 90%, the lipid yield was 93% and the content of DAG in lipid was 1.2%. Other factors are optimized using the central combination design experimental methods, and the design and results are shown in Table 2. As seen from Table 2, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

### Embodiment 3

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 0.10 g/kg, and reaction duration of 4.0 h to hydrolysis pectin. Thirdly, the soybean slurry was transferred in the third order of three-phase magnetic fluidized bed reactor to continue the reaction. The mixture was adjusted pH to 9.5, temperature of 55° C. The reaction of the third order is: nano-immobilized magnetic alkaline protease dosage of 0.12 g/kg, the reaction duration of 4.0 h. Fourthly, the soybean slurry was transferred in the fourth order of three-phase magnetic fluidized bed reactor to continue the reaction. The mixture was adjusted pH to 6.5, temperature of 60° C., immobilized phospholipase A1 dosage of 0.30 g/kg, and reaction duration of 3.4 h. Fifthly, the above soybean slurry was heated to 60° C. followed by adjusting pH to 7.0 using a buffer, and then the mixture was transferred in the fifth order of three-phase fluidized bed reactor to generate DAG from PC and PE. The reaction of the fifth order is: nano-immobilized magnetic phospholipase C dosage of 0.90 g/kg, the reaction duration of 4.5 h. After completion of all other reaction steps, the mixture from the fifth order of three-phase fluidized bed reactor was centrifugated to obtain lipid, skim and residues. In the total skim, the protein yield was 91%, the lipid yield was 94% and the content of DAG in lipid was 1.1%. Other factors are optimized using the central combination design experimental methods, and the design and results are shown in Table 3. As seen from Table 3, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

### Embodiment 4

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 0.10 g/kg, and reaction duration of 4.0 h to hydrolysis pectin. Thirdly, the soybean slurry was transferred in the third order of three-phase magnetic fluidized bed reactor to continue the reaction. The mixture was adjusted pH to 6.5, temperature of 60° C., immobilized phospholipase A1 dosage of 0.20 g/kg, and reaction duration of 3.4 h. Fourthly, the above soybean slurry was heated to 60° C. followed by adjusting pH to 7.0 using a buffer, and then the mixture was transferred in the fourth order of three-phase fluidized bed reactor to generate DAG from PC and PE. The reaction of the fourth order is: nano-immobilized magnetic phospholipase C dosage of 0.90 g/kg, the reaction duration of 4.5 h. Fifthly, the soybean slurry was transferred in the fifth order of three-phase magnetic fluidized bed reactor to continue the reaction. The mixture was adjusted pH to 9.5, temperature of 55° C. The reaction of the fifth order is: nano-immobilized magnetic alkaline protease dosage of 0.90 g/kg, the reaction duration of 4.0 h. After completion of all other reaction steps, the mixture from the fifth order of three-phase fluidized bed reactor was centrifugated to obtain lipid, skim and residues. In the total skim, the protein yield was 90%, the lipid yield was 92% and the content of DAG in lipid was 1.3%. Other factors are optimized using the central combination design experimental methods, and the design and results are shown in Table 4. As seen from Table 4, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

### Embodiment 5

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 9.6 mg/kg, and. reaction duration of 4.0 h to hydrolysis pectin. Thirdly, the soybean slurry was transferred in the third order of three-phase magnetic fluidized bed reactor to continue the reaction. The mixture was adjusted pH to 9.5, temperature of 55° C. The reaction of the third order is: nano-immobilized magnetic alkaline protease dosage of 0.10 g/kg, the reaction duration of 4.0 h. Fourthly, the above soybean slurry was heated to 60° C. followed by adjusting pH to 7.0 using a buffer, and then the mixture was transferred in the fourth order of three-phase fluidized bed reactor to generate DAG from PC and PE. The reaction of the fourth order is: nano-immobilized magnetic phospholipase C dosage of 0.70 g/kg, the reaction duration of 4.5 h. Fifthly, the soybean slurry was transferred in the fifth order of three-phase magnetic fluidized bed reactor to continue the reaction. The mixture was adjusted pH to 6.5, temperature of 60° C., immobilized phospholipase A1 dosage of 0.30 g/kg, and reaction duration of 3.4 h. After completion of all other reaction steps, the mixture from the fifth order of three-phase fluidized bed reactor was centrifugated to obtain lipid, skim and residues. In the total skim, the protein yield was 95%, the lipid yield was 97% and the content of DAG in lipid was 1.6%. Other factors are optimized using the central combination design experimental methods, and the design and results are shown in Table 5. As seen from Table 5, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

### Embodiment 6

The present embodiment is to develop a method for nano-magnetic immobilized enzymes to continuously and directionally catalyze soybean slurry for the extraction of protein and functional lipid, which are disclosed in the present invention. Firstly, the soybean slurry was heated to 50° C. followed by adjusting pH to 4.5 using a buffer, and then the mixture was transferred in the first order of three-phase fluidized bed reactor. The reaction of the first order is: nano-immobilized magnetic cellulose dosage of 0.12 g/kg, the reaction duration of 4.0 h. Secondly, the soybean slurry was transferred in the second order of three-phase magnetic fluidized bed reactor to continue the reaction. The mixture was adjusted pH to 3.5, temperature of 55° C., immobilized pectinase dosage of 0.10 g/kg, and reaction duration of 4.0 h to hydrolysis pectin. Thirdly, the soybean slurry was transferred in the third order of three-phase magnetic fluidized bed reactor to continue the reaction. The mixture was adjusted pH to 9.5, temperature of 55° C. The reaction of the third order is: nano-immobilized magnetic alkaline protease dosage of 0.10 g/kg, the reaction duration of 4.0 h. Fourthly, the soybean slurry was transferred in the fourth order of three-phase magnetic fluidized bed reactor to continue the reaction. The mixture was adjusted pH to 6,5, temperature of 60° C., immobilized phospholipase A1 dosage of 0.20 g/kg, and reaction duration of 3.4 h. Fifthly, the above soybean slurry was heated to 60° C. followed by adjusting pH to 7.0 using a buffer, and then the mixture was transferred in the fifth order of three-phase fluidized bed reactor to generate DAG from PC and PE. The reaction of the fifth order is: nano-immobilized magnetic phospholipase C dosage of 0.80 g/kg, the reaction duration of 4.5 h. After completion of all other reaction steps, the mixture from the fifth order of three-phase fluidized bed reactor was centrifugated to obtain lipid, skim and residues. In the total skim, the protein yield was 94%, the lipid yield was 95% and the content of DAG in lipid was 1.5%. Other factors are optimized using the central combination design experimental methods, and the design and results are shown in Table 6. As seen from Table 6, with increasing the reaction temperature, the lipid and protein yield are decreased, indicating that the temperature has detrimental effect on the activity of the nano-magnetic immobilized enzyme, with lowered enzymatic efficiency and thereby decreasing the content of DAG in lipid. With increasing pH, the yield of functional lipid and protein decreased, with reduced content of DAG. With extending the reaction duration, the yield of lipid is stayed at a similar level with no significant changes in the content of DAG. Therefore, the best optimization configuration is shown in Group 5.

TABLE 1

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | temper- | | Enzyme | | | | Enzyme | | | | Enzyme | | | | Enzyme | | the | the | of DAG in |
| | ature | | amount | time | ature | | amount | time | Temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 96 | 94 | 1.5 |
| 2 | 40 | 4.5 | 0.12 | 4.0 | 55 | 3.5 | 0.10 | 4.0 | 55 | 9.5 | 0.10 | 4.0 | 50 | 7.0 | 0.80 | 4.5 | 60 | 6.5 | 0.40 | 3.4 | 93 | 92 | 1.4 |
| 3 | 60 | 4.5 | 0.12 | 4.0 | 55 | 3.5 | 0.10 | 4.0 | 55 | 9.5 | 0.10 | 4.0 | 70 | 7.0 | 0.80 | 4.5 | 60 | 6.5 | 0.40 | 3.4 | 92 | 91 | 1.4 |
| 4 | 50 | 3.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 91 | 92 | 1.3 |
| 5 | 50 | 5.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 93 | 91 | 1.3 |
| 6 | 50 | 4.5 | 0.08 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 92 | 91 | 1.4 |
| 7 | 50 | 4.5 | 0.14 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 91 | 92 | 1.3 |
| 8 | 50 | 4.5 | 0.12 | 3.5 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 91 | 92 | 1.4 |
| 9 | 50 | 4.5 | 0.12 | 5.5 | 55 | 3.5 | 0.10 | 4.0 | 55 | 9.5 | 0.10 | 4.0 | 60 | 7.0 | 0.80 | 4.5 | 60 | 6.5 | 0.40 | 3.4 | 92 | 91 | 1.3 |
| 10 | 50 | 4.5 | 0.12 | 4.0 | 45 | 3.5 | 0.10 | 4.0 | 55 | 9.5 | 0.10 | 4.0 | 60 | 7.0 | 0.80 | 4.5 | 60 | 6.5 | 0.40 | 3.4 | 93 | 92 | 1.4 |
| 11 | 50 | 4.5 | 0.12 | 4.0 | 65 | 3.5 | 0.10 | 4.0 | 55 | 9.5 | 0.10 | 4.0 | 60 | 7.0 | 0.80 | 4.5 | 60 | 6.5 | 0.40 | 3.4 | 92 | 91 | 1.4 |
| 12 | 50 | 4.5 | 0.12 | 4.0 | 55 | 2.5 | 0.10 | 4.0 | 55 | 9.5 | 0.10 | 4.0 | 60 | 7.0 | 0.80 | 4.5 | 60 | 6.5 | 0.40 | 3.4 | 93 | 92 | 1.4 |
| 13 | 50 | 4.5 | 0.12 | 4.0 | 55 | 4.5 | 0.10 | 4.0 | 55 | 9.5 | 0.10 | 4.0 | 60 | 7.0 | 0.80 | 4.5 | 60 | 6.5 | 0.40 | 3.4 | 91 | 92 | 1.3 |
| 14 | 50 | 4.5 | 0.12 | 4.0 | 55 | 3.5 | 0.08 | 4.0 | 55 | 9.5 | 0.10 | 4.0 | 60 | 7.0 | 0.80 | 4.5 | 60 | 6.5 | 0.40 | 3.4 | 92 | 92 | 1.3 |
| 15 | 50 | 4.5 | 0.12 | 4.0 | 55 | 3.5 | 0.12 | 4.0 | 55 | 9.5 | 0.10 | 4.0 | 60 | 7.0 | 0.80 | 4.5 | 60 | 6.5 | 0.40 | 3.4 | 93 | 91 | 1.4 |
| 16 | 50 | 4.5 | 0.12 | 4.0 | 55 | 3.5 | 0.10 | 3.0 | 55 | 9.5 | 0.10 | 4.0 | 60 | 7.0 | 0.80 | 4.5 | 60 | 6.5 | 0.40 | 3.4 | 91 | 91 | 1.3 |
| 17 | 50 | 4.5 | 0.12 | 4.0 | 55 | 3.5 | 0.10 | 5.0 | 55 | 9.5 | 0.10 | 4.0 | 60 | 7.0 | 0.80 | 4.5 | 60 | 6.5 | 0.40 | 3.4 | 92 | 92 | 1.4 |
| 18 | 50 | 4.5 | 0.12 | 4.0 | 55 | 3.5 | 0.10 | 4.0 | 45 | 9.5 | 0.10 | 4.0 | 60 | 7.0 | 0.80 | 4.5 | 60 | 6.5 | 0.40 | 3.4 | 93 | 91 | 1.4 |
| 19 | 50 | 4.5 | 0.12 | 4.0 | 55 | 3.5 | 0.10 | 4.0 | 65 | 9.5 | 0.10 | 4.0 | 60 | 7.0 | 0.80 | 4.5 | 60 | 6.5 | 0.40 | 3.4 | 92 | 92 | 1.4 |
| 20 | 50 | 4.5 | 0.12 | 4.0 | 55 | 3.5 | 0.10 | 4.0 | 55 | 8.5 | 0.10 | 4.0 | 60 | 7.0 | 0.80 | 4.5 | 60 | 6.5 | 0.40 | 3.4 | 91 | 91 | 1.3 |
| 21 | 50 | 4.5 | 0.12 | 4.0 | 55 | 3.5 | 0.10 | 4.0 | 55 | 10.5 | 0.10 | 4.0 | 60 | 7.0 | 0.80 | 4.5 | 60 | 6.5 | 0.40 | 3.4 | 91 | 92 | 1.3 |
| 22 | 50 | 4.5 | 0.12 | 4.0 | 55 | 3.5 | 0.10 | 4.0 | 55 | 9.5 | 0.08 | 4.0 | 60 | 7.0 | 0.80 | 4.5 | 60 | 6.5 | 0.40 | 3.4 | 92 | 91 | 1.4 |
| 23 | 50 | 4.5 | 0.12 | 4.0 | 55 | 3.5 | 0.10 | 4.0 | 55 | 9.5 | 0.12 | 4.0 | 60 | 7.0 | 0.80 | 4.5 | 60 | 6.5 | 0.40 | 3.4 | 93 | 91 | 1.3 |
| 24 | 50 | 4.5 | 0.12 | 4.0 | 55 | 3.5 | 0.10 | 4.0 | 55 | 9.5 | 0.10 | 3.0 | 60 | 7.0 | 0.80 | 4.5 | 60 | 6.5 | 0.40 | 3.4 | 93 | 92 | 1.4 |
| 25 | 50 | 4.5 | 0.12 | 4.0 | 55 | 3.5 | 0.10 | 4.0 | 55 | 9.5 | 0.10 | 5.0 | 60 | 7.0 | 0.80 | 4.5 | 60 | 6.5 | 0.40 | 3.4 | 91 | 92 | 1.3 |
| 26 | 50 | 4.5 | 0.12 | 4.0 | 55 | 3.5 | 0.10 | 4.0 | 55 | 9.5 | 0.10 | 4.0 | 50 | 7.0 | 0.80 | 4.5 | 60 | 6.5 | 0.40 | 3.4 | 92 | 92 | 1.4 |
| 27 | 50 | 4.5 | 0.12 | 4.0 | 55 | 3.5 | 0.10 | 4.0 | 55 | 9.5 | 0.10 | 4.0 | 70 | 7.0 | 0.80 | 4.5 | 60 | 6.5 | 0.40 | 3.4 | 92 | 91 | 1.4 |
| 28 | 50 | 4.5 | 0.12 | 4.0 | 55 | 3.5 | 0.10 | 4.0 | 55 | 9.5 | 0.10 | 4.0 | 60 | 6.0 | 0.80 | 4.5 | 60 | 6.5 | 0.40 | 3.4 | 91 | 91 | 1.3 |
| 29 | 50 | 4.5 | 0.12 | 4.0 | 55 | 3.5 | 0.10 | 4.0 | 55 | 9.5 | 0.10 | 4.0 | 60 | 8.0 | 0.80 | 4.5 | 60 | 6.5 | 0.40 | 3.4 | 91 | 92 | 1.3 |
| 30 | 50 | 4.5 | 0.12 | 4.0 | 55 | 3.5 | 0.10 | 4.0 | 55 | 9.5 | 0.10 | 4.0 | 60 | 7.0 | 0.90 | 4.5 | 60 | 6.5 | 0.40 | 3.4 | 91 | 91 | 1.4 |
| 31 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.40 | 3.4 | 93 | 92 | 1.3 |
| 32 | 50 | 4.5 | 0.12 | 4.0 | 55 | 3.5 | 0.10 | 4.0 | 55 | 9.5 | 0.10 | 4.0 | 60 | 7.0 | 0.80 | 4.0 | 60 | 6.5 | 0.40 | 3.4 | 91 | 92 | 1.4 |
| 33 | 50 | 4.5 | 0.12 | 4.0 | 55 | 3.5 | 0.10 | 4.0 | 55 | 9.5 | 0.10 | 4.0 | 60 | 7.0 | 0.80 | 5.0 | 60 | 6.5 | 0.40 | 3.4 | 92 | 92 | 1.3 |
| 34 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 50 | 6.5 | 0.40 | 3.4 | 92 | 91 | 1.4 |
| 35 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 70 | 6.5 | 0.40 | 3.4 | 91 | 91 | 1.4 |
| 36 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.0 | 0.40 | 3.4 | 91 | 92 | 1.4 |
| 37 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 7.0 | 0.40 | 3.4 | 91 | 91 | 1.3 |
| 38 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.50 | 3.4 | 93 | 92 | 1.3 |
| 39 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.30 | 3.4 | 92 | 91 | 1.4 |
| 40 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.2 | 92 | 92 | 1.3 |
| 41 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.40 | 3.6 | 91 | 91 | 1.4 |

TABLE 2

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | temper- | | Enzyme | | | | Enzyme | | | | Enzyme | | | | Enzyme | | the | the | of DAG in |
| | ature | | amount | time | ature | | amount | time | Temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 93 | 90 | 1.2 |
| 2 | 40 | 4.5 | 0.12 | 4.0 | 55 | 3.5 | 0.10 | 4.0 | 50 | 7.0 | 0.90 | 4.5 | 60 | 6.5 | 0.50 | 3.4 | 55 | 9.5 | 0.12 | 4.0 | 92 | 87 | 1.1 |
| 3 | 60 | 4.5 | 0.12 | 4.0 | 55 | 3.5 | 0.10 | 4.0 | 70 | 7.0 | 0.90 | 4.5 | 60 | 6.5 | 0.50 | 3.4 | 55 | 9.5 | 0.12 | 4.0 | 91 | 89 | 1.0 |
| 4 | 50 | 3.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 92 | 88 | 1.1 |
| 5 | 50 | 5.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 91 | 89 | 1.0 |
| 6 | 50 | 4.5 | 0.08 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 91 | 88 | 1.0 |
| 7 | 50 | 4.5 | 0.14 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 92 | 86 | 1.1 |
| 8 | 50 | 4.5 | 0.12 | 3.5 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 92 | 85 | 1.0 |
| 9 | 50 | 4.5 | 0.12 | 5.5 | 55 | 3.5 | 0.10 | 4.0 | 60 | 7.0 | 0.90 | 4.5 | 60 | 6.5 | 0.50 | 3.4 | 55 | 9.5 | 0.12 | 4.0 | 91 | 87 | 1.1 |
| 10 | 50 | 4.5 | 0.12 | 4.0 | 45 | 3.5 | 0.10 | 4.0 | 60 | 7.0 | 0.90 | 4.5 | 60 | 6.5 | 0.50 | 3.4 | 55 | 9.5 | 0.12 | 4.0 | 92 | 86 | 1.1 |
| 11 | 50 | 4.5 | 0.12 | 4.0 | 65 | 3.5 | 0.10 | 4.0 | 60 | 7.0 | 0.90 | 4.5 | 60 | 6.5 | 0.50 | 3.4 | 55 | 9.5 | 0.12 | 4.0 | 91 | 87 | 1.0 |
| 12 | 50 | 4.5 | 0.12 | 4.0 | 55 | 2.5 | 0.10 | 4.0 | 60 | 7.0 | 0.90 | 4.5 | 60 | 6.5 | 0.50 | 3.4 | 55 | 9.5 | 0.12 | 4.0 | 92 | 88 | 1.1 |
| 13 | 50 | 4.5 | 0.12 | 4.0 | 55 | 4.5 | 0.10 | 4.0 | 60 | 7.0 | 0.90 | 4.5 | 60 | 6.5 | 0.50 | 3.4 | 55 | 9.5 | 0.12 | 4.0 | 92 | 86 | 1.0 |
| 14 | 50 | 4.5 | 0.12 | 4.0 | 55 | 3.5 | 0.08 | 4.0 | 60 | 7.0 | 0.90 | 4.5 | 60 | 6.5 | 0.50 | 3.4 | 55 | 9.5 | 0.12 | 4.0 | 92 | 87 | 1.0 |
| 15 | 50 | 4.5 | 0.12 | 4.0 | 55 | 3.5 | 0.12 | 4.0 | 60 | 7.0 | 0.90 | 4.5 | 60 | 6.5 | 0.50 | 3.4 | 55 | 9.5 | 0.12 | 4.0 | 91 | 88 | 1.1 |
| 16 | 50 | 4.5 | 0.12 | 4.0 | 55 | 3.5 | 0.10 | 3.0 | 60 | 7.0 | 0.90 | 4.5 | 60 | 6.5 | 0.50 | 3.4 | 55 | 9.5 | 0.12 | 4.0 | 91 | 86 | 1.0 |
| 17 | 50 | 4.5 | 0.12 | 4.0 | 55 | 3.5 | 0.10 | 5.0 | 60 | 7.0 | 0.90 | 4.5 | 60 | 6.5 | 0.50 | 3.4 | 55 | 9.5 | 0.12 | 4.0 | 92 | 87 | 1.1 |
| 18 | 50 | 4.5 | 0.12 | 4.0 | 55 | 3.5 | 0.10 | 4.0 | 50 | 7.0 | 0.90 | 4.5 | 60 | 6.5 | 0.50 | 3.4 | 55 | 9.5 | 0.12 | 4.0 | 91 | 86 | 1.1 |
| 19 | 50 | 4.5 | 0.12 | 4.0 | 55 | 3.5 | 0.10 | 4.0 | 70 | 7.0 | 0.90 | 4.5 | 60 | 6.5 | 0.50 | 3.4 | 55 | 9.5 | 0.12 | 4.0 | 92 | 88 | 1.0 |
| 20 | 50 | 4.5 | 0.12 | 4.0 | 55 | 3.5 | 0.10 | 4.0 | 60 | 6.0 | 0.90 | 4.5 | 60 | 6.5 | 0.50 | 3.4 | 55 | 9.5 | 0.12 | 4.0 | 91 | 86 | 1.1 |
| 21 | 50 | 4.5 | 0.12 | 4.0 | 55 | 3.5 | 0.10 | 4.0 | 60 | 8.0 | 0.90 | 4.5 | 60 | 6.5 | 0.50 | 3.4 | 55 | 9.5 | 0.12 | 4.0 | 92 | 87 | 1.0 |
| 22 | 50 | 4.5 | 0.12 | 4.0 | 55 | 3.5 | 0.10 | 4.0 | 60 | 7.0 | 1.00 | 4.5 | 60 | 6.5 | 0.50 | 3.4 | 55 | 9.5 | 0.12 | 4.0 | 91 | 88 | 1.0 |
| 23 | 50 | 4.5 | 0.12 | 4.0 | 55 | 3.5 | 0.10 | 4.0 | 60 | 7.0 | 0.80 | 4.5 | 60 | 6.5 | 0.50 | 3.4 | 55 | 9.5 | 0.12 | 4.0 | 91 | 86 | 1.1 |
| 24 | 50 | 4.5 | 0.12 | 4.0 | 55 | 3.5 | 0.10 | 4.0 | 60 | 7.0 | 0.90 | 4.0 | 60 | 6.5 | 0.50 | 3.4 | 55 | 9.5 | 0.12 | 4.0 | 92 | 88 | 1.0 |
| 25 | 50 | 4.5 | 0.12 | 4.0 | 55 | 3.5 | 0.10 | 4.0 | 60 | 7.0 | 0.90 | 5.0 | 60 | 6.5 | 0.50 | 3.4 | 55 | 9.5 | 0.12 | 4.0 | 92 | 87 | 1.1 |
| 26 | 50 | 4.5 | 0.12 | 4.0 | 55 | 3.5 | 0.10 | 4.0 | 60 | 7.0 | 0.90 | 4.5 | 50 | 6.5 | 0.50 | 3.4 | 55 | 9.5 | 0.12 | 4.0 | 92 | 87 | 1.1 |
| 27 | 50 | 4.5 | 0.12 | 4.0 | 55 | 3.5 | 0.10 | 4.0 | 60 | 7.0 | 0.90 | 4.5 | 70 | 6.5 | 0.50 | 3.4 | 55 | 9.5 | 0.12 | 4.0 | 91 | 88 | 1.0 |
| 28 | 50 | 4.5 | 0.12 | 4.0 | 55 | 3.5 | 0.10 | 4.0 | 60 | 7.0 | 0.90 | 4.5 | 60 | 6.0 | 0.50 | 3.4 | 55 | 9.5 | 0.12 | 4.0 | 91 | 86 | 1.1 |
| 29 | 50 | 4.5 | 0.12 | 4.0 | 55 | 3.5 | 0.10 | 4.0 | 60 | 7.0 | 0.90 | 4.5 | 60 | 7.0 | 0.50 | 3.4 | 55 | 9.5 | 0.12 | 4.0 | 92 | 87 | 1.0 |
| 30 | 50 | 4.5 | 0.12 | 4.0 | 55 | 3.5 | 0.10 | 4.0 | 60 | 7.0 | 0.90 | 4.5 | 60 | 6.5 | 0.60 | 3.4 | 55 | 9.5 | 0.12 | 4.0 | 91 | 86 | 1.0 |
| 31 | 50 | 4.5 | 0.12 | 4.0 | 55 | 3.5 | 0.10 | 4.0 | 60 | 7.0 | 0.90 | 4.5 | 60 | 6.5 | 0.40 | 3.4 | 55 | 9.5 | 0.12 | 4.0 | 92 | 85 | 1.1 |
| 32 | 50 | 4.5 | 0.12 | 4.0 | 55 | 3.5 | 0.10 | 4.0 | 60 | 7.0 | 0.90 | 4.5 | 60 | 6.5 | 0.50 | 3.2 | 55 | 9.5 | 0.12 | 4.0 | 92 | 86 | 1.0 |
| 33 | 50 | 4.5 | 0.12 | 4.0 | 55 | 3.5 | 0.10 | 4.0 | 60 | 7.0 | 0.90 | 4.5 | 60 | 6.5 | 0.50 | 3.6 | 55 | 9.5 | 0.12 | 4.0 | 92 | 84 | 1.1 |
| 34 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 50 | 9.5 | 0.12 | 4.0 | 91 | 87 | 1.1 |
| 35 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 60 | 9.5 | 0.12 | 4.0 | 91 | 86 | 1.0 |
| 36 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.0 | 0.12 | 4.0 | 92 | 86 | 1.1 |
| 37 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 10. | 0.12 | 4.0 | 91 | 87 | 1.0 |
| 38 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.14 | 4.0 | 92 | 86 | 1.0 |
| 39 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.10 | 4.0 | 91 | 85 | 1.1 |
| 40 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 3.5 | 92 | 84 | 1.0 |
| 41 | 50 | 4.5 | 0.12 | 4.0 | 55 | 3.5 | 0.10 | 4.0 | 60 | 7.0 | 0.90 | 4.5 | 60 | 6.5 | 0.50 | 3.4 | 55 | 9.5 | 0.12 | 4.5 | 91 | 85 | 1.1 |

TABLE 3

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | Tem- | | Enzyme | | | | Enzyme | | | | Enzyme | | | | Enzyme | | the | the | of DAG in |
| | ature | | amount | time | perature | | amount | time | Temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 7.0 | 0.90 | 4.5 | 94 | 91 | 1.1 |
| 2 | 40 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 93 | 92 | 1.0 |
| 3 | 60 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 92 | 91 | 0.9 |
| 4 | 50 | 3.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 91 | 92 | 0.9 |
| 5 | 50 | 5.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 93 | 91 | 1.0 |
| 6 | 50 | 4.5 | 0.08 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 92 | 91 | 0.9 |
| 7 | 50 | 4.5 | 0.14 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 91 | 92 | 1.0 |
| 8 | 50 | 4.5 | 0.12 | 3.5 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 91 | 92 | 0.9 |
| 9 | 50 | 4.5 | 0.12 | 5.5 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 92 | 91 | 1.0 |
| 10 | 50 | 4.5 | 0.12 | 4.0 | 45 | 3.5 | 0.10 | 4.0 | 55 | 9.5 | 0.10 | 4.0 | 60 | 6.5 | 0.30 | 3.4 | 60 | 6.5 | 0.90 | 4.5 | 93 | 92 | 0.9 |
| 11 | 50 | 4.5 | 0.12 | 4.0 | 65 | 3.5 | 0.10 | 4.0 | 55 | 9.5 | 0.10 | 4.0 | 60 | 6.5 | 0.30 | 3.4 | 60 | 6.5 | 0.90 | 4.5 | 92 | 91 | 1.0 |
| 12 | 50 | 4.5 | 0.12 | 4.0 | 55 | 2.5 | 0.10 | 4.0 | 55 | 9.5 | 0.10 | 4.0 | 60 | 6.5 | 0.30 | 3.4 | 60 | 6.5 | 0.90 | 4.5 | 93 | 92 | 1.0 |
| 13 | 50 | 4.5 | 0.12 | 4.0 | 55 | 4.5 | 0.10 | 4.0 | 55 | 9.5 | 0.10 | 4.0 | 60 | 6.5 | 0.30 | 3.4 | 60 | 6.5 | 0.90 | 4.5 | 91 | 92 | 0.9 |
| 14 | 50 | 4.5 | 0.12 | 4.0 | 55 | 3.5 | 0.08 | 4.0 | 55 | 9.5 | 0.10 | 4.0 | 60 | 6.5 | 0.30 | 3.4 | 60 | 6.5 | 0.90 | 4.5 | 92 | 92 | 0.9 |
| 15 | 50 | 4.5 | 0.12 | 4.0 | 55 | 3.5 | 0.12 | 4.0 | 55 | 9.5 | 0.10 | 4.0 | 60 | 6.5 | 0.30 | 3.4 | 60 | 6.5 | 0.90 | 4.5 | 93 | 91 | 1.0 |
| 16 | 50 | 4.5 | 0.12 | 4.0 | 55 | 3.5 | 0.10 | 3.0 | 55 | 9.5 | 0.10 | 4.0 | 60 | 6.5 | 0.30 | 3.4 | 60 | 6.5 | 0.90 | 4.5 | 91 | 91 | 0.9 |
| 17 | 50 | 4.5 | 0.12 | 4.0 | 55 | 3.5 | 0.10 | 5.0 | 55 | 9.5 | 0.10 | 4.0 | 60 | 6.5 | 0.30 | 3.4 | 60 | 6.5 | 0.90 | 4.5 | 92 | 92 | 1.0 |
| 18 | 50 | 4.5 | 0.12 | 4.0 | 55 | 3.5 | 0.10 | 4.0 | 45 | 9.5 | 0.10 | 4.0 | 60 | 6.5 | 0.30 | 3.4 | 60 | 6.5 | 0.90 | 4.5 | 93 | 91 | 0.9 |
| 19 | 50 | 4.5 | 0.12 | 4.0 | 55 | 3.5 | 0.10 | 4.0 | 65 | 9.5 | 0.10 | 4.0 | 60 | 6.5 | 0.30 | 3.4 | 60 | 6.5 | 0.90 | 4.5 | 92 | 92 | 1.0 |
| 20 | 50 | 4.5 | 0.12 | 4.0 | 55 | 3.5 | 0.10 | 4.0 | 55 | 8.5 | 0.10 | 4.0 | 60 | 6.5 | 0.30 | 3.4 | 60 | 6.5 | 0.90 | 4.5 | 91 | 91 | 0.9 |
| 21 | 50 | 4.5 | 0.12 | 4.0 | 55 | 3.5 | 0.10 | 4.0 | 55 | 10.5 | 0.10 | 4.0 | 60 | 6.5 | 0.30 | 3.4 | 60 | 6.5 | 0.90 | 4.5 | 91 | 92 | 1.0 |
| 22 | 50 | 4.5 | 0.12 | 4.0 | 55 | 3.5 | 0.10 | 4.0 | 55 | 9.5 | 0.08 | 4.0 | 60 | 6.5 | 0.30 | 3.4 | 60 | 6.5 | 0.90 | 4.5 | 92 | 91 | 1.0 |
| 23 | 50 | 4.5 | 0.12 | 4.0 | 55 | 3.5 | 0.10 | 4.0 | 55 | 9.5 | 0.12 | 4.0 | 60 | 6.5 | 0.30 | 3.4 | 60 | 6.5 | 0.90 | 4.5 | 93 | 91 | 0.9 |
| 24 | 50 | 4.5 | 0.12 | 4.0 | 55 | 3.5 | 0.10 | 4.0 | 55 | 9.5 | 0.10 | 3.0 | 60 | 6.5 | 0.30 | 3.4 | 60 | 6.5 | 0.90 | 4.5 | 93 | 92 | 0.9 |
| 25 | 50 | 4.5 | 0.12 | 4.0 | 55 | 3.5 | 0.10 | 4.0 | 55 | 9.5 | 0.10 | 5.0 | 60 | 6.5 | 0.30 | 3.4 | 60 | 6.5 | 0.90 | 4.5 | 91 | 92 | 1.0 |
| 26 | 50 | 4.5 | 0.12 | 4.0 | 55 | 3.5 | 0.10 | 4.0 | 55 | 9.5 | 0.10 | 4.0 | 50 | 6.5 | 0.30 | 3.4 | 60 | 6.5 | 0.90 | 4.5 | 92 | 92 | 0.9 |
| 27 | 50 | 4.5 | 0.12 | 4.0 | 55 | 3.5 | 0.10 | 4.0 | 55 | 9.5 | 0.10 | 4.0 | 70 | 6.5 | 0.30 | 3.4 | 60 | 6.5 | 0.90 | 4.5 | 92 | 91 | 1.0 |
| 28 | 50 | 4.5 | 0.12 | 4.0 | 55 | 3.5 | 0.10 | 4.0 | 55 | 9.5 | 0.10 | 4.0 | 60 | 6.0 | 0.30 | 3.4 | 60 | 6.5 | 0.90 | 4.5 | 91 | 91 | 0.9 |
| 29 | 50 | 4.5 | 0.12 | 4.0 | 55 | 3.5 | 0.10 | 4.0 | 55 | 9.5 | 0.10 | 4.0 | 60 | 7.0 | 0.30 | 3.4 | 60 | 6.5 | 0.90 | 4.5 | 91 | 92 | 1.0 |
| 30 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.5 | 91 | 91 | 0.9 |
| 31 | 50 | 4.5 | 0.12 | 4.0 | 55 | 3.5 | 0.10 | 4.0 | 55 | 9.5 | 0.10 | 4.0 | 60 | 6.5 | 0.40 | 3.4 | 60 | 6.5 | 0.90 | 4.5 | 93 | 92 | 1.0 |
| 32 | 50 | 4.5 | 0.12 | 4.0 | 55 | 3.5 | 0.10 | 4.0 | 55 | 9.5 | 0.10 | 4.0 | 60 | 6.5 | 0.20 | 3.2 | 60 | 6.5 | 0.90 | 4.5 | 91 | 92 | 1.0 |
| 33 | 50 | 4.5 | 0.12 | 4.0 | 55 | 3.5 | 0.10 | 4.0 | 55 | 9.5 | 0.10 | 4.0 | 60 | 6.5 | 0.30 | 3.6 | 60 | 6.5 | 0.90 | 4.5 | 92 | 92 | 0.9 |
| 34 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 50 | 6.5 | 0.90 | 4.5 | 92 | 91 | 0.9 |
| 35 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 70 | 6.5 | 0.90 | 4.5 | 91 | 91 | 1.0 |
| 36 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.0 | 0.90 | 4.5 | 91 | 92 | 0.9 |
| 37 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 7.0 | 0.90 | 4.5 | 91 | 91 | 1.0 |
| 38 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 1.00 | 4.5 | 93 | 92 | 0.9 |
| 39 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.80 | 4.5 | 92 | 91 | 1.0 |
| 40 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 5.0 | 92 | 92 | 0.9 |
| 41 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.90 | 4.0 | 91 | 91 | 1.0 |

TABLE 4

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | temper- | | Enzyme | | | | Enzyme | | | | Enzyme | | | | Enzyme | | the | the | of DAG in |
| | ature | | amount | time | ature | | amount | time | temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.12 | 4.0 | 92 | 90 | 1.3 |
| 2 | 40 | 4.5 | 0.12 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 90 | 87 | 1.2 |
| 3 | 60 | 4.5 | 0.12 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 91 | 89 | 1.1 |
| 4 | 50 | 3.5 | 0.12 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 90 | 88 | 1.0 |
| 5 | 50 | 5.5 | 0.12 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 9 | 89 | 1.2 |
| 6 | 50 | 4.5 | 0.08 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 91 | 88 | 1.1 |
| 7 | 50 | 4.5 | 0.14 | 4.0 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 89 | 86 | 1.2 |
| 8 | 50 | 4.5 | 0.12 | 3.5 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 89 | 85 | 1.0 |
| 9 | 50 | 4.5 | 0.12 | 5.5 | 55 | 3.5 | 0.10 | 4.0 | 60 | 6.5 | 0.20 | 3.4 | 60 | 6.5 | 0.90 | 4.5 | 55 | 9.5 | 0.12 | 4.0 | 91 | 87 | 1.2 |
| 10 | 50 | 4.5 | 0.12 | 4.0 | 45 | 3.5 | 0.10 | 4.0 | 60 | 6.5 | 0.20 | 3.4 | 60 | 6.5 | 0.90 | 4.5 | 55 | 9.5 | 0.12 | 4.0 | 89 | 86 | 1.1 |
| 11 | 50 | 4.5 | 0.12 | 4.0 | 65 | 3.5 | 0.10 | 4.0 | 60 | 6.5 | 0.20 | 3.4 | 60 | 6.5 | 0.90 | 4.5 | 55 | 9.5 | 0.12 | 4.0 | 91 | 87 | 1.1 |
| 12 | 50 | 4.5 | 0.12 | 4.0 | 55 | 2.5 | 0.10 | 4.0 | 60 | 6.5 | 0.20 | 3.4 | 60 | 6.5 | 0.90 | 4.5 | 55 | 9.5 | 0.12 | 4.0 | 89 | 88 | 1.2 |
| 13 | 50 | 4.5 | 0.12 | 4.0 | 55 | 4.5 | 0.10 | 4.0 | 60 | 6.5 | 0.20 | 3.4 | 60 | 6.5 | 0.90 | 4.5 | 55 | 9.5 | 0.12 | 4.0 | 89 | 86 | 1.1 |
| 14 | 50 | 4.5 | 0.12 | 4.0 | 55 | 3.5 | 0.08 | 4.0 | 60 | 6.5 | 0.20 | 3.4 | 60 | 6.5 | 0.90 | 4.5 | 55 | 9.5 | 0.12 | 4.0 | 89 | 87 | 1.0 |
| 15 | 50 | 4.5 | 0.12 | 4.0 | 55 | 3.5 | 0.12 | 4.0 | 60 | 6.5 | 0.20 | 3.4 | 60 | 6.5 | 0.90 | 4.5 | 55 | 9.5 | 0.12 | 4.0 | 91 | 88 | 1.2 |
| 16 | 50 | 4.5 | 0.12 | 4.0 | 55 | 3.5 | 0.10 | 3.0 | 60 | 6.5 | 0.20 | 3.4 | 60 | 6.5 | 0.90 | 4.5 | 55 | 9.5 | 0.12 | 4.0 | 91 | 86 | 1.1 |
| 17 | 50 | 4.5 | 0.12 | 4.0 | 55 | 3.5 | 0.10 | 5.0 | 60 | 6.5 | 0.20 | 3.4 | 60 | 6.5 | 0.90 | 4.5 | 55 | 9.5 | 0.12 | 4.0 | 89 | 87 | 1.2 |
| 18 | 50 | 4.5 | 0.12 | 4.0 | 55 | 3.5 | 0.10 | 4.0 | 50 | 6.5 | 0.20 | 3.4 | 60 | 6.5 | 0.90 | 4.5 | 55 | 9.5 | 0.12 | 4.0 | 90 | 86 | 1.0 |
| 19 | 50 | 4.5 | 0.12 | 4.0 | 55 | 3.5 | 0.10 | 4.0 | 70 | 6.5 | 0.20 | 3.4 | 60 | 6.5 | 0.90 | 4.5 | 55 | 9.5 | 0.12 | 4.0 | 89 | 88 | 1.2 |
| 20 | 50 | 4.5 | 0.12 | 4.0 | 55 | 3.5 | 0.10 | 4.0 | 60 | 6.0 | 0.20 | 3.4 | 60 | 6.5 | 0.90 | 4.5 | 55 | 9.5 | 0.12 | 4.0 | 90 | 86 | 1.1 |
| 21 | 50 | 4.5 | 0.12 | 4.0 | 55 | 3.5 | 0.10 | 4.0 | 60 | 7.0 | 0.20 | 3.4 | 60 | 6.5 | 0.90 | 4.5 | 55 | 9.5 | 0.12 | 4.0 | 89 | 87 | 1.1 |
| 22 | 50 | 4.5 | 0.12 | 4.0 | 55 | 3.5 | 0.10 | 4.0 | 60 | 6.5 | 0.30 | 3.4 | 60 | 6.5 | 0.90 | 4.5 | 55 | 9.5 | 0.12 | 4.0 | 89 | 88 | 1.2 |
| 23 | 50 | 4.5 | 0.12 | 4.0 | 55 | 3.5 | 0.10 | 4.0 | 60 | 6.5 | 0.40 | 3.4 | 60 | 6.5 | 0.90 | 4.5 | 55 | 9.5 | 0.12 | 4.0 | 90 | 86 | 1.1 |
| 24 | 50 | 4.5 | 0.12 | 4.0 | 55 | 3.5 | 0.10 | 4.0 | 60 | 6.5 | 0.20 | 3.2 | 60 | 6.5 | 0.90 | 4.5 | 55 | 9.5 | 0.12 | 4.0 | 89 | 88 | 1.0 |
| 25 | 50 | 4.5 | 0.12 | 4.0 | 55 | 3.5 | 0.10 | 4.0 | 60 | 6.5 | 0.20 | 3.6 | 60 | 6.5 | 0.90 | 4.5 | 55 | 9.5 | 0.12 | 4.0 | 90 | 87 | 1.2 |
| 26 | 50 | 4.5 | 0.12 | 4.0 | 55 | 3.5 | 0.10 | 4.0 | 50 | 6.5 | 0.20 | 3.4 | 50 | 6.5 | 0.90 | 4.5 | 55 | 9.5 | 0.12 | 4.0 | 90 | 87 | 1.1 |
| 27 | 50 | 4.5 | 0.12 | 4.0 | 55 | 3.5 | 0.10 | 4.0 | 70 | 6.5 | 0.20 | 3.4 | 70 | 6.5 | 0.90 | 4.5 | 55 | 9.5 | 0.12 | 4.0 | 89 | 88 | 1.2 |
| 28 | 50 | 4.5 | 0.12 | 4.0 | 55 | 3.5 | 0.10 | 4.0 | 60 | 6.5 | 0.20 | 3.4 | 60 | 6.0 | 0.90 | 4.5 | 55 | 9.5 | 0.12 | 4.0 | 89 | 86 | 1.0 |
| 29 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.12 | 4.0 | 90 | 87 | 1.2 |
| 30 | 50 | 4.5 | 0.12 | 4.0 | 55 | 3.5 | 0.10 | 4.0 | 60 | 6.5 | 0.20 | 3.4 | 60 | 6.5 | 1.00 | 4.5 | 55 | 9.5 | 0.12 | 4.0 | 89 | 86 | 1.1 |
| 31 | 50 | 4.5 | 0.12 | 4.0 | 55 | 3.5 | 0.10 | 4.0 | 60 | 6.5 | 0.20 | 3.4 | 60 | 6.5 | 0.80 | 4.5 | 55 | 9.5 | 0.12 | 4.0 | 90 | 85 | 1.1 |
| 32 | 50 | 4.5 | 0.12 | 4.0 | 55 | 3.5 | 0.10 | 4.0 | 60 | 6.5 | 0.20 | 3.4 | 60 | 6.5 | 0.90 | 5.0 | 55 | 9.5 | 0.12 | 4.0 | 90 | 86 | 1.2 |
| 33 | 50 | 4.5 | 0.12 | 4.0 | 55 | 3.5 | 0.10 | 4.0 | 60 | 6.5 | 0.20 | 3.4 | 60 | 6.5 | 0.90 | 4.0 | 55 | 9.5 | 0.12 | 4.0 | 89 | 84 | 1.1 |
| 34 | 50 | 4.5 | 0.12 | 4.0 | 55 | 3.5 | 0.10 | 4.0 | 50 | 6.5 | 0.20 | 3.4 | 60 | 6.5 | 0.90 | 4.5 | 50 | 9.5 | 0.12 | 4.0 | 90 | 87 | 1.0 |
| 35 | 50 | 4.5 | 0.12 | 4.0 | 55 | 3.5 | 0.10 | 4.0 | 70 | 6.5 | 0.20 | 3.4 | 60 | 6.5 | 0.90 | 4.5 | 60 | 9.5 | 0.12 | 4.0 | 89 | 86 | 1.2 |
| 36 | 50 | 4.5 | 0.12 | 4.0 | 55 | 3.5 | 0.10 | 4.0 | 60 | 6.5 | 0.20 | 3.4 | 60 | 6.5 | 0.90 | 4.5 | 55 | 9.0 | 0.12 | 4.0 | 90 | 86 | 1.1 |
| 37 | 50 | 4.5 | 0.12 | 4.0 | 55 | 3.5 | 0.10 | 4.0 | 60 | 6.5 | 0.20 | 3.4 | 60 | 6.5 | 0.90 | 4.5 | 55 | 10. | 0.12 | 4.0 | 90 | 87 | 1.2 |
| 38 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.14 | 4.0 | 89 | 86 | 1.0 |
| 39 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.10 | 4.0 | 89 | 85 | 1.2 |
| 40 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.12 | 3.5 | 90 | 84 | 1.1 |
| 41 | 50 | 4.5 | 0.12 | 4.0 | 55 | 3.5 | 0.10 | 4.0 | 60 | 6.5 | 0.20 | 3.4 | 60 | 7.0 | 0.90 | 4.5 | 55 | 9.5 | 0.12 | 4.5 | 90 | 85 | 1.1 |

TABLE 5

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | Tem- | | Enzyme | | | | Enzyme | | | | Enzyme | | | | Enzyme | | the | the | of DAG in |
| | ature | | amount | time | perature | | amount | time | Temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 97 | 95 | 1.6 |
| 2 | 40 | 4.5 | 0.12 | 4.0 | 55 | 3.5 | 0.10 | 4.0 | 55 | 9.5 | 0.10 | 4.0 | 50 | 7.0 | 0.70 | 4.5 | 60 | 6.5 | 0.30 | 3.4 | 94 | 93 | 1.4 |
| 3 | 60 | 4.5 | 0.12 | 4.0 | 55 | 3.5 | 0.10 | 4.0 | 55 | 9.5 | 0.10 | 4.0 | 70 | 7.0 | 0.70 | 4.5 | 60 | 6.5 | 0.30 | 3.4 | 95 | 93 | 1.4 |
| 4 | 50 | 3.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 93 | 92 | 1.5 |
| 5 | 50 | 5.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 93 | 93 | 1.3 |
| 6 | 50 | 4.5 | 0.08 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 92 | 94 | 1.4 |
| 7 | 50 | 4.5 | 0.14 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 93 | 92 | 1.5 |
| 8 | 50 | 4.5 | 0.12 | 3.5 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 94 | 93 | 1.4 |
| 9 | 50 | 4.5 | 0.12 | 6.5 | 55 | 3.5 | 0.10 | 4.0 | 55 | 9.5 | 0.10 | 4.0 | 60 | 7.0 | 0.70 | 4.5 | 60 | 6.5 | 0.30 | 3.4 | 92 | 92 | 1.3 |
| 10 | 50 | 4.5 | 0.12 | 4.0 | 45 | 3.5 | 0.10 | 4.0 | 55 | 9.5 | 0.10 | 4.0 | 60 | 7.0 | 0.70 | 4.5 | 60 | 6.5 | 0.30 | 3.4 | 93 | 93 | 1.4 |
| 11 | 50 | 4.5 | 0.12 | 4.0 | 65 | 3.5 | 0.10 | 4.0 | 55 | 9.5 | 0.10 | 4.0 | 60 | 7.0 | 0.70 | 4.5 | 60 | 6.5 | 0.30 | 3.4 | 92 | 94 | 1.5 |
| 12 | 50 | 4.5 | 0.12 | 4.0 | 55 | 2.5 | 0.10 | 4.0 | 55 | 9.5 | 0.10 | 4.0 | 60 | 7.0 | 0.70 | 4.5 | 60 | 6.5 | 0.30 | 3.4 | 93 | 94 | 1.4 |
| 13 | 50 | 4.5 | 0.12 | 4.0 | 55 | 4.5 | 0.10 | 4.0 | 55 | 9.5 | 0.10 | 4.0 | 60 | 7.0 | 0.70 | 4.5 | 60 | 6.5 | 0.30 | 3.4 | 94 | 93 | 1.3 |
| 14 | 50 | 4.5 | 0.12 | 4.0 | 55 | 3.5 | 0.08 | 4.0 | 55 | 9.5 | 0.10 | 4.0 | 60 | 7.0 | 0.70 | 4.5 | 60 | 6.5 | 0.30 | 3.4 | 94 | 93 | 1.3 |
| 15 | 50 | 4.5 | 0.12 | 4.0 | 55 | 3.5 | 0.12 | 4.0 | 55 | 9.5 | 0.10 | 4.0 | 60 | 7.0 | 0.70 | 4.5 | 60 | 6.5 | 0.30 | 3.4 | 95 | 92 | 1.4 |
| 16 | 50 | 4.5 | 0.12 | 4.0 | 55 | 3.5 | 0.10 | 3.0 | 55 | 9.5 | 0.10 | 4.0 | 60 | 7.0 | 0.70 | 4.5 | 60 | 6.5 | 0.30 | 3.4 | 93 | 93 | 1.3 |
| 17 | 50 | 4.5 | 0.12 | 4.0 | 55 | 3.5 | 0.10 | 5.0 | 55 | 9.5 | 0.10 | 4.0 | 60 | 7.0 | 0.70 | 4.5 | 60 | 6.5 | 0.30 | 3.4 | 93 | 94 | 1.5 |
| 18 | 50 | 4.5 | 0.12 | 4.0 | 55 | 3.5 | 0.10 | 4.0 | 45 | 9.5 | 0.10 | 4.0 | 60 | 7.0 | 0.70 | 4.5 | 60 | 6.5 | 0.30 | 3.4 | 92 | 92 | 1.4 |
| 19 | 50 | 4.5 | 0.12 | 4.0 | 55 | 3.5 | 0.10 | 4.0 | 65 | 9.5 | 0.10 | 4.0 | 60 | 7.0 | 0.70 | 4.5 | 60 | 6.5 | 0.30 | 3.4 | 93 | 93 | 1.4 |
| 20 | 50 | 4.5 | 0.12 | 4.0 | 55 | 3.5 | 0.10 | 4.0 | 55 | 8.5 | 0.10 | 4.0 | 60 | 7.0 | 0.70 | 4.5 | 60 | 6.5 | 0.30 | 3.4 | 94 | 92 | 1.3 |
| 21 | 50 | 4.5 | 0.12 | 4.0 | 55 | 3.5 | 0.10 | 4.0 | 55 | 10.5 | 0.10 | 4.0 | 60 | 7.0 | 0.70 | 4.5 | 60 | 6.5 | 0.30 | 3.4 | 92 | 93 | 1.3 |
| 22 | 50 | 4.5 | 0.12 | 4.0 | 55 | 3.5 | 0.10 | 4.0 | 55 | 9.5 | 0.08 | 4.0 | 60 | 7.0 | 0.70 | 4.5 | 60 | 6.5 | 0.30 | 3.4 | 93 | 94 | 1.4 |
| 23 | 50 | 4.5 | 0.12 | 4.0 | 55 | 3.5 | 0.10 | 4.0 | 55 | 9.5 | 0.12 | 4.0 | 60 | 7.0 | 0.70 | 4.5 | 60 | 6.5 | 0.30 | 3.4 | 92 | 94 | 1.5 |
| 24 | 50 | 4.5 | 0.12 | 4.0 | 55 | 3.5 | 0.10 | 4.0 | 55 | 9.5 | 0.10 | 3.0 | 60 | 7.0 | 0.70 | 4.5 | 60 | 6.5 | 0.30 | 3.4 | 93 | 93 | 1.4 |
| 25 | 50 | 4.5 | 0.12 | 4.0 | 55 | 3.5 | 0.10 | 4.0 | 55 | 9.5 | 0.10 | 5.0 | 60 | 7.0 | 0.70 | 4.5 | 60 | 6.5 | 0.30 | 3.4 | 94 | 93 | 1.5 |
| 26 | 50 | 4.5 | 0.12 | 4.0 | 55 | 3.5 | 0.10 | 4.0 | 55 | 9.5 | 0.10 | 4.0 | 50 | 7.0 | 0.70 | 4.5 | 60 | 6.5 | 0.30 | 3.4 | 94 | 92 | 1.4 |
| 27 | 50 | 4.5 | 0.12 | 4.0 | 55 | 3.5 | 0.10 | 4.0 | 55 | 9.5 | 0.10 | 4.0 | 70 | 7.0 | 0.70 | 4.5 | 60 | 6.5 | 0.30 | 3.4 | 92 | 93 | 1.4 |
| 28 | 50 | 4.5 | 0.12 | 4.0 | 55 | 3.5 | 0.10 | 4.0 | 55 | 9.5 | 0.10 | 4.0 | 60 | 6.0 | 0.70 | 4.5 | 60 | 6.5 | 0.30 | 3.4 | 94 | 94 | 1.3 |
| 29 | 50 | 4.5 | 0.12 | 4.0 | 55 | 3.5 | 0.10 | 4.0 | 55 | 9.5 | 0.10 | 4.0 | 60 | 8.0 | 0.70 | 4.5 | 60 | 6.5 | 0.30 | 3.4 | 95 | 92 | 1.3 |
| 30 | 50 | 4.5 | 0.12 | 4.0 | 55 | 3.5 | 0.10 | 4.0 | 55 | 9.5 | 0.10 | 4.0 | 60 | 7.0 | 0.80 | 4.5 | 60 | 6.5 | 0.30 | 3.4 | 93 | 93 | 1.4 |
| 31 | 50 | 4.5 | 0.12 | 4.0 | 55 | 3.5 | 0.10 | 4.0 | 55 | 9.5 | 0.10 | 4.0 | 60 | 7.0 | 0.60 | 4.5 | 60 | 6.5 | 0.30 | 3.4 | 93 | 92 | 1.3 |
| 32 | 50 | 4.5 | 0.12 | 4.0 | 55 | 3.5 | 0.10 | 4.0 | 55 | 9.5 | 0.10 | 4.0 | 60 | 7.0 | 0.70 | 4.0 | 60 | 6.5 | 0.30 | 3.4 | 92 | 93 | 1.4 |
| 33 | 50 | 4.5 | 0.12 | 4.0 | 55 | 3.5 | 0.10 | 4.0 | 55 | 9.5 | 0.10 | 4.0 | 60 | 7.0 | 0.70 | 5.0 | 60 | 6.5 | 0.30 | 3.4 | 93 | 94 | 1.3 |
| 34 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 50 | 6.5 | 0.30 | 3.4 | 94 | 94 | 1.4 |
| 35 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 70 | 6.5 | 0.30 | 3.4 | 92 | 93 | 1.5 |
| 36 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.0 | 0.30 | 3.4 | 93 | 93 | 1.4 |
| 37 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 7.0 | 0.30 | 3.4 | 92 | 92 | 1.5 |
| 38 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.40 | 3.4 | 93 | 93 | 1.4 |
| 39 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.20 | 3.4 | 92 | 94 | 1.5 |
| 40 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.2 | 92 | 92 | 1.4 |
| 41 | 50 | 4.5 | 0.12 | 4.0 | 55 | 3.5 | 0.10 | 4.0 | 55 | 9.5 | 0.10 | 4.0 | 60 | 7.0 | 0.70 | 4.5 | 60 | 6.5 | 0.30 | 3.6 | 94 | 93 | 1.4 |

TABLE 6

| | nano-magnetic | | | | nano-magnetic immobilized | | | | nano-magnetic immobilized | | | | nano-magnetic immobilized | | | | nano-magnetic immobilized | | | | | | |
|---|---|---|---|---|---|---|---|---|---|---|---|---|---|---|---|---|---|---|---|---|---|---|---|
| | immobilized cellulase | | | | pectinase | | | | phospholipase C | | | | phospholipase A1 | | | | alkaline protease | | | | | | the content |
| | temper- | | Enzyme | | Tem- | | Enzyme | | | | Enzyme | | | | Enzyme | | | | Enzyme | | the | the | of DAG in |
| | ature | | amount | time | perature | | amount | time | Temperature | | amount | time | temperature | | amount | time | temperature | | amount | time | lipidyield | proteinyield | lipid |
| | (° C.) | pH | g/Kg | (h) | (° C.) | pH | g/Kg | (h) | (° C.) | pH | g/Kg | (h) | (° C.) | pH | g/Kg | (h) | (° C.) | pH | g/Kg | (h) | (%) | (%) | (%) |
| 1 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 7.0 | 0.80 | 4.5 | 95 | 94 | 1.5 |
| 2 | 40 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 93 | 92 | 1.4 |
| 3 | 60 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 92 | 91 | 1.4 |
| 4 | 50 | 3.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 94 | 92 | 1.3 |
| 5 | 50 | 5.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 93 | 93 | 1.3 |
| 6 | 50 | 4.5 | 0.08 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 92 | 93 | 1.4 |
| 7 | 50 | 4.5 | 0.14 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 94 | 92 | 1.3 |
| 8 | 50 | 4.5 | 0.12 | 3.5 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 94 | 92 | 1.4 |
| 9 | 50 | 4.5 | 0.12 | 6.5 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 92 | 91 | 1.3 |
| 10 | 50 | 4.5 | 0.12 | 4.0 | 45 | 3.5 | 0.10 | 4.0 | 55 | 9.5 | 0.10 | 4.0 | 60 | 6.5 | 0.20 | 3.4 | 60 | 6.5 | 0.80 | 4.5 | 93 | 92 | 1.4 |
| 11 | 50 | 4.5 | 0.12 | 4.0 | 65 | 3.5 | 0.10 | 4.0 | 55 | 9.5 | 0.10 | 4.0 | 60 | 6.5 | 0.20 | 3.4 | 60 | 6.5 | 0.80 | 4.5 | 92 | 91 | 1.4 |
| 12 | 50 | 4.5 | 0.12 | 4.0 | 55 | 2.5 | 0.10 | 4.0 | 55 | 9.5 | 0.10 | 4.0 | 60 | 6.5 | 0.20 | 3.4 | 60 | 6.5 | 0.80 | 4.5 | 93 | 92 | 1.4 |
| 13 | 50 | 4.5 | 0.12 | 4.0 | 55 | 4.5 | 0.10 | 4.0 | 55 | 9.5 | 0.10 | 4.0 | 60 | 6.5 | 0.20 | 3.4 | 60 | 6.5 | 0.80 | 4.5 | 94 | 92 | 1.2 |
| 14 | 50 | 4.5 | 0.12 | 4.0 | 55 | 3.5 | 0.08 | 4.0 | 55 | 9.5 | 0.10 | 4.0 | 60 | 6.5 | 0.20 | 3.4 | 60 | 6.5 | 0.80 | 4.5 | 92 | 92 | 1.2 |
| 15 | 50 | 4.5 | 0.12 | 4.0 | 55 | 3.5 | 0.14 | 4.0 | 55 | 9.5 | 0.10 | 4.0 | 60 | 6.5 | 0.20 | 3.4 | 60 | 6.5 | 0.80 | 4.5 | 93 | 91 | 1.0 |
| 16 | 50 | 4.5 | 0.12 | 4.0 | 55 | 3.5 | 0.10 | 3.0 | 55 | 9.5 | 0.10 | 4.0 | 60 | 6.5 | 0.20 | 3.4 | 60 | 6.5 | 0.80 | 4.5 | 91 | 91 | 1.2 |
| 17 | 50 | 4.5 | 0.12 | 4.0 | 55 | 3.5 | 0.10 | 5.0 | 55 | 9.5 | 0.10 | 4.0 | 60 | 6.5 | 0.20 | 3.4 | 60 | 6.5 | 0.80 | 4.5 | 92 | 92 | 1.2 |
| 18 | 50 | 4.5 | 0.12 | 4.0 | 55 | 3.5 | 0.10 | 4.0 | 45 | 9.5 | 0.10 | 4.0 | 60 | 6.5 | 0.20 | 3.4 | 60 | 6.5 | 0.80 | 4.5 | 93 | 91 | 1.4 |
| 19 | 50 | 4.5 | 0.12 | 4.0 | 55 | 3.5 | 0.10 | 4.0 | 65 | 9.5 | 0.10 | 4.0 | 60 | 6.5 | 0.20 | 3.4 | 60 | 6.5 | 0.80 | 4.5 | 92 | 92 | 1.3 |
| 20 | 50 | 4.5 | 0.12 | 4.0 | 55 | 3.5 | 0.10 | 4.0 | 55 | 8.5 | 0.10 | 4.0 | 60 | 6.5 | 0.20 | 3.4 | 60 | 6.5 | 0.80 | 4.5 | 94 | 91 | 1.3 |
| 21 | 50 | 4.5 | 0.12 | 4.0 | 55 | 3.5 | 0.10 | 4.0 | 55 | 10.5 | 0.10 | 4.0 | 60 | 6.5 | 0.20 | 3.4 | 60 | 6.5 | 0.80 | 4.5 | 94 | 92 | 1.4 |
| 22 | 50 | 4.5 | 0.12 | 4.0 | 55 | 3.5 | 0.10 | 4.0 | 55 | 9.5 | 0.08 | 4.0 | 60 | 6.5 | 0.20 | 3.4 | 60 | 6.5 | 0.80 | 4.5 | 92 | 93 | 1.3 |
| 23 | 50 | 4.5 | 0.12 | 4.0 | 55 | 3.5 | 0.10 | 4.0 | 55 | 9.5 | 0.12 | 4.0 | 60 | 6.5 | 0.20 | 3.4 | 60 | 6.5 | 0.80 | 4.5 | 93 | 93 | 1.4 |
| 24 | 50 | 4.5 | 0.12 | 4.0 | 55 | 3.5 | 0.10 | 4.0 | 55 | 9.5 | 0.10 | 3.0 | 60 | 6.5 | 0.20 | 3.4 | 60 | 6.5 | 0.80 | 4.5 | 93 | 92 | 1.3 |
| 25 | 50 | 4.5 | 0.12 | 4.0 | 55 | 3.5 | 0.10 | 4.0 | 55 | 9.5 | 0.10 | 5.0 | 60 | 6.5 | 0.20 | 3.4 | 60 | 6.5 | 0.80 | 4.5 | 91 | 92 | 1.4 |
| 26 | 50 | 4.5 | 0.12 | 4.0 | 55 | 3.5 | 0.10 | 4.0 | 55 | 9.5 | 0.10 | 4.0 | 50 | 6.5 | 0.20 | 3.4 | 60 | 6.5 | 0.80 | 4.5 | 92 | 92 | 1.4 |
| 27 | 50 | 4.5 | 0.12 | 4.0 | 55 | 3.5 | 0.10 | 4.0 | 55 | 9.5 | 0.10 | 4.0 | 70 | 6.5 | 0.20 | 3.4 | 60 | 6.5 | 0.80 | 4.5 | 92 | 93 | 1.2 |
| 28 | 50 | 4.5 | 0.12 | 4.0 | 55 | 3.5 | 0.10 | 4.0 | 55 | 9.5 | 0.10 | 4.0 | 60 | 6.0 | 0.20 | 3.4 | 60 | 6.5 | 0.80 | 4.5 | 94 | 93 | 1.2 |
| 29 | 50 | 4.5 | 0.12 | 4.0 | 55 | 3.5 | 0.10 | 4.0 | 55 | 9.5 | 0.10 | 4.0 | 60 | 7.0 | 0.20 | 3.4 | 60 | 6.5 | 0.80 | 4.5 | 94 | 92 | 1.3 |
| 30 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.5 | 91 | 91 | 1.3 |
| 31 | 50 | 4.5 | 0.12 | 4.0 | 55 | 3.5 | 0.10 | 4.0 | 55 | 9.5 | 0.10 | 4.0 | 60 | 6.5 | 0.30 | 3.4 | 60 | 6.5 | 0.80 | 4.5 | 93 | 92 | 1.4 |
| 32 | 50 | 4.5 | 0.12 | 4.0 | 55 | 3.5 | 0.10 | 4.0 | 55 | 9.5 | 0.10 | 4.0 | 60 | 6.5 | 0.10 | 3.2 | 60 | 6.5 | 0.80 | 4.5 | 94 | 92 | 1.3 |
| 33 | 50 | 4.5 | 0.12 | 4.0 | 55 | 3.5 | 0.10 | 4.0 | 55 | 9.5 | 0.10 | 4.0 | 60 | 6.5 | 0.20 | 3.6 | 60 | 6.5 | 0.80 | 4.5 | 92 | 92 | 1.4 |
| 34 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 50 | 6.5 | 0.80 | 4.5 | 92 | 93 | 1.3 |
| 35 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 70 | 6.5 | 0.80 | 4.5 | 94 | 93 | 1.3 |
| 36 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.0 | 0.80 | 4.5 | 94 | 92 | 1.3 |
| 37 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 7.0 | 0.80 | 4.5 | 94 | 93 | 1.4 |
| 38 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.90 | 4.5 | 93 | 92 | 1.3 |
| 39 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.70 | 4.5 | 92 | 93 | 1.3 |
| 40 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 5.0 | 92 | 92 | 1.4 |
| 41 | 50 | 4.5 | 0.12 | 4.0 | 55 | 3.5 | 0.10 | 4.0 | 55 | 9.5 | 0.10 | 4.0 | 60 | 6.5 | 0.20 | 3.4 | 60 | 6.5 | 0.80 | 4.0 | 94 | 93 | 1.3 |

What is claimed is:

1. A method for extracting proteins and functional lipid from soybean liquid by directional continuous catalysis with nano magnetic immobilized enzymes, comprising following steps:
adding the nano magnetic immobilized enzymes in a five-order-three-phase magnetic fluidized bed and taking the nano magnetic immobilized enzymes as a stationary phase;
taking a soybean as a mobile phase, and enabling the nano magnetic immobilized enzymes in a five-order-three-phase magnetic fluidized bed reaction system to be steady by adjusting reaction operation parameters and performing directional enzymolysis reaction with corresponding zymolyte;
increasing enzymolysis effect by adjusting a sequence and reaction conditions based on different enzymolysis properties of the nano magnetic immobilized enzymes;
obtaining a soy protein and functional oils with rich DAG and realizing extraction of proteins and functional lipid from soybean liquid by directional continuous catalysis with nano magnetic immobilized enzymes;
wherein the five-order-three-phase fluidized bed reaction system comprises a three-phase (gas-liquid-solid) magnetic fluidized bed, a soybean material tank, a pH regulating tank, and a power plant;
wherein the nano magnetic immobilized enzymes comprises nano magnetic immobilized cellulase, nano magnetic immobilized pectinase, nano magnetic immobilized protease, nano magnetic immobilized phospholipase C and nano magnetic immobilized phospholipase A1.

2. The method for extracting proteins and functional lipid from soybean liquid by directional continuous catalysis with nano magnetic immobilized enzymes according to claim 1, wherein the method comprises following specific steps:
heating the soybean slurry 50° C., adding buffer and adjusting pH to 4.5;
transferring the soybean slurry to a first tank of the five-order-three-phase fluidized bed reaction system with nano-immobilized magnetic cellulose dosage of 0.12 g/kg; reacting for 4.0 h for enzymolysis of cellulose in the soybean liquid and discharging the soybean liquid from the first tank;
mixing the discharged soybean liquid with the buffer, adjusting pH value to 3.5, transferring to a mixer for mixing, heating to 55° C. and transferring to a second tank of the five-order-three-phase fluidized bed reaction system with nano-immobilized pectinase dosage of 0.10 g/kg, reacting for 4.0 h for enzymolysis of pectinase in the soybean liquid and discharging the soybean liquid from the second tank;
transferring soybean slurry to the third tank with adding nano-immobilized alkaline protease at the dosage of 0.10 g/kg, adjusting pH to 9.5, temperature of 55° C., and reaction duration of 4.0 h;
transferring soybean slurry to the fourth tank with adding nano-immobilized phospholipase C at the dosage of 0.80 g/kg, adjusting pH to 7.0, temperature of 60° C., and reaction duration of 4.5 h to generate DAG from PC and PE;
transferring soybean slurry to the fifth tank with adding nano-immobilized phospholipase A1 at the dosage of 0.40 g/kg, adjusting pH to 6.5, temperature of 60° C., and reaction duration of 3.4 h to generate DAG from PC and PE;
centrifuging to obtain lipid, skim and residue, with the protein yield of 94% (by mass of skim), the lipid yield of 96% (by mass of skim) and the content of DAG in lipid was 1.5%.

3. The method for extracting proteins and functional lipid from soybean liquid by directional continuous catalysis with nano magnetic immobilized enzymes according to claim 1, wherein the method comprises following specific steps:
heating the soybean liquid to 50° C. adding buffer and adjusting pH to 4.5; transferring the soybean slurry to the first tank of the five-order-three-phase fluidized bed reaction system with nano-immobilized magnetic cellulose dosage of 0.12 g/kg; reacting for 4.0 h. for enzymolysis of cellulose in the soybean liquid and discharging the soybean liquid from the first tank; mixing the discharged soybean slurry with the buffer, adjusting pH value to 3.5, to a mixer for mixing, heating to 55° C. and transferring to a second tank of the five-order-three-phase fluidized bed reaction system with nano-immobilized pectinase dosage of 0.10 g/kg reacting for 4.0 h for enzymolysis of pectinase in the soybean liquid and discharging the soybean liquid from the second tank;
transferring soybean slurry to the third tank with adding nano-immobilized phospholipase C at the dosage of 0.90 g/kg, adjusting pH to 7.0, temperature of 60° C. and reaction duration of 4.5 h to generate DAG from PC and PE;
transferring soybean slurry to the fourth tank with adding nano-immobilized phospholipase A1 at the dosage of 0.50 g/kg, adjusting pH to 6.5, temperature of 60° C., and reaction duration of 3.4 h to generate DAG from PC and PE;
transferring soybean slurry to the fifth tank with adding nano-immobilized alkaline protease at the dosage of 0.12 g/kg, adjusting pH to 9.5, temperature of 55° C., and reaction duration of 4.0 h;
centrifuging to obtain lipid, skim and residue, with the protein yield of 90% (by mass of skim), the lipid yield of 93% (by mass of skim) and the content of DAG in lipid was 1.2%.

4. The method for extracting proteins and functional lipid from soybean liquid by directional continuous catalysis with nano magnetic immobilized enzymes according to claim 1, wherein the method comprises following specific steps: heating the soybean slurry 50° C., adding buffer and adjusting pH to 4.5;
transferring the soybean liquid to the first tank of the five-order-three-phase fluidized bed reaction system with nano-immobilized magnetic cellulose dosage of 0.12 g/kg; reacting for 4.0 h for enzymolysis of cellulose in the soybean liquid and discharging the soybean liquid from the first tank;
mixing the discharged soybean slurry with the buffer, adjusting pH value to 3.5, transferring to a mixer for mixing, heating to 55° C. and transferring to a second tank of the five-order-three-phase fluidized bed reaction system with nano-immobilized pectinase at the dosage of 0.10 g/kg, reacting for 4.0 h for enzymolysis of pectinase in the soybean liquid and discharging the soybean liquid from the second tank; transferring soybean slurry to the third tank with adding nano-immobilized alkaline protease at the dosage of 0.12 g/kg, adjusting pH to 9.5, temperature of 55° C., and reaction duration of 4.0 h;
transferring soybean slurry to the fourth tank with adding nano-immobilized phospholipase A1 at the dosage of 0.30 g/kg, adjusting pH to 6.5, temperature of 60° C., and reaction duration of 3.4 h to generate DAG from PC and PE;
transferring soybean slurry to the fifth tank with adding nano-immobilized phospholipase C at the dosage of 0.90 g/kg, adjusting pH to 7.0, temperature of 60° C., and reaction duration of 4.5 h to generate DAG from PC and PE;
centrifuging to obtain lipid, skim and residue, with the protein yield of 91% (by mass of skim), the lipid yield of 94% (by mass of skim) and the content of DAG in lipid was 1.1%.

5. The method for extracting proteins and functional lipid from soybean liquid by directional continuous catalysis with nano magnetic immobilized enzymes according to claim 1, wherein the method comprises following specific steps:
heating the soybean slurry 50° C., adding buffer and adjusting pH to 4.5;
transferring soybean slurry to the first tank of the five-order-three-phase fluidized bed reaction system with nano-immobilized magnetic cellulose dosage of 0.12 g/kg; reacting for 4.0 h for enzymolysis of cellulose in the soybean liquid and discharging the soybean liquid from the first tank;
mixing the discharged soybean slurry with the buffer adjusting pH value to 3.5, transferring to a mixer for mixing, heating to 55° C. and transferring to a second tank of the five-order-three-phase fluidized bed reaction system with nano-immobilized pectinase at the dosage of 0.10 g/kg, reacting for 4.0 h for enzymolysis of pectinase in the soybean liquid and discharging the soybean liquid from the second tank;
transferring soybean slurry to the third tank with adding nano-immobilized phospholipase A1 at the dosage of 0.20 g/kg, adjusting pH to 6.5, temperature of 60° C., and reaction duration of 3.4 h to generate DAG from PC and PE;
transferring soybean slurry to the fourth tank with adding nano-immobilized phospholipase C at the dosage of 0.90 g/kg, adjusting pH to 7.0, temperature of 60° C., and reaction duration of 4.5 h to generate DAG from PC and PE;
transferring soybean slurry to the fifth tank with adding nano-immobilized alkaline protease at the dosage of 0.90 g/kg, adjusting pH to 9.5, temperature of 55° C., and reaction duration of 4.0 h;
centrifuging to obtain lipid, skim and residue, with the protein yield of 90% (by mass of skim), the lipid yield of 92% (by mass of skim) and the content of DAG in lipid was 1.3%.

6. The method for extracting proteins and functional lipid from soybean liquid by directional continuous catalysis with nano magnetic immobilized enzymes according to claim 1, wherein the method comprises following specific steps:
heating the soybean slurry 50° C., adding buffer and adjusting pH to 4.5;
transferring soybean slurry to the first tank of the five-order-three-phase fluidized bed reaction system with nano-immobilized magnetic cellulose dosage of 0.12 g/kg; reacting for 4.0 h for enzymolysis of cellulose in the soybean liquid and discharging the soybean liquid from the first tank;
mixing the discharged soybean slurry with the buffer, adjusting pH value to 3.5, transferring to a mixer for mixing, heating to 55° C. and transferring soybean slurry to the second tank with adding nano-immobilized pectinase at the dosage of 9.6 mg/kg, reacting for 4.0 h for enzymolysis of pectinase in the soybean liquid and discharging the soybean liquid from the second tank;
transferring soybean slurry to the third tank with adding nano-immobilized alkaline protease at the dosage of 0.10 g/kg, adjusting pH to 9.5, temperature of 55° C., and reaction duration of 4.0 h;
transferring soybean slurry to the fourth tank with adding nano-immobilized phospholipase C at the dosage of 0.70 g/kg, adjusting pH to 7.0, temperature of 60° C. and reaction duration of 4.5 h to generate DAG from PC and PE;
transferring the soybean slurry to the fifth tank with adding nano-immobilized phospholipase A1 at the dosage of 0.30 g/kg, adjusting pH to 6.5, temperature of 60° C., and reaction duration of 3.4 h to generate DAG from PC and PE;
centrifuging to obtain lipid, skim and residue, with the protein yield of 95% (by mass of skim), the lipid yield of 97% (by mass of skim) and the content of DAG in lipid was 1.6%.

7. The method for extracting proteins and functional lipid from soybean liquid by directional continuous catalysis with nano magnetic immobilized enzymes according to claim 1, wherein the method comprises following specific steps:
heating the soybean slurry 50° C., adding buffer and adjusting pH to 4.5;
transferring the soybean slurry to the first tank of the five-order-three-phase fluidized bed reaction system with nano-immobilized magnetic cellulose dosage of 0.12 g/kg; reacting for 4.0 h for enzymolysis of cellulose in the soybean liquid and discharging the soybean liquid from the first tank;
mixing the discharged soybean slurry with the buffer, adjusting pH value to 3.5, transferring to a mixer for mixing, heating to 55° C. and transferring to a second tank of the five-order-three-phase fluidized bed reaction system with nano-immobilized pectinase at the dosage of 0.10 g/kg, reacting for 4.0 h for enzymolysis of pectinase in the soybean liquid and discharging the soybean liquid from the second tank; transferring soybean slurry to the third tank with adding nano-immobilized alkaline protease at the dosage of 0.10 g/kg, adjusting pH to 9.5, temperature of 55° C., and reaction duration of 4.0 h;
transferring soybean slurry to the fourth tank with adding nano-immobilized phospholipase A1 at the dosage of 0.20 g/kg, adjusting pH to 6.5, temperature of 60° C., and reaction duration of 3.4 h to generate DAG from PC and PE;
transferring soybean slurry to the fifth tank with adding nano-immobilized phospholipase C at the dosage of 0.80 g/kg, adjusting pH to 7.0, temperature of 60° C., and reaction duration of 4.5 h to generate DAG from PC and PE;
centrifuging to obtain lipid, skim and residue, with the protein yield of 94% (by mass of skim), the lipid yield of 95% (by mass of skim) and the content of DAG in lipid was 1.5%.
